(19) United States
(12) Patent Application Publication
(10) Pub. No.: US 2013/0164227 A1
(43) Pub. Date: Jun. 27, 2013

(54) PRODUCTION METHOD AND EFFERVESCENT FORMULATIONS COMPRISING CEPHALOSPORIN AND CLAVULANIC ACID

(71) Applicant: Mahmut Bilgic, Istanbul (TR)

(72) Inventor: Mahmut Bilgic, Istanbul (TR)

(21) Appl. No.: 13/692,138

(22) Filed: Dec. 3, 2012

Related U.S. Application Data
(63) Continuation-in-part of application No. PCT/TR2011/000149, filed on Jun. 2, 2011.
Application No. PCT/TR2011/000149 is a continuation-in-part of application No. PCT/TR2011/000146, filed on Jun. 2, 2011.

(30) Foreign Application Priority Data
Jun. 3, 2010 (TR) TR 2010/04462

(51) Int. Cl. A61K 47/10

(52) U.S. Cl. CPC A61K 47/10
USPC 424/44; 241/24.1; 264/115

(57) ABSTRACT

The present invention relates to the process for the preparation of the pharmaceutical formulations comprising a cephalosporin antibiotic and clavulanic acid or any pharmaceutically acceptable derivative thereof. The present invention also relates to processes for preparation of said formulations and their use in bacterial infections.

### CROSS-REFERENCE TO RELATED APPLICATIONS

This application is a continuation-in-part of PCT/TR2011/000146 and PCT/TR2011/000149, filed Jun. 2, 2011, which are incorporated herein by reference in their entireties. This application is entitled to and claims priority benefits to application Serial Number TR2010/04462, filed Jun. 3, 2010.

### BACKGROUND OF THE INVENTION

Like other beta-lactam antibiotics, cephalosporin induces bactericide effect. However, some types of bacteria such as Escherichia, Klebsiella, Proteus, Pseudomonas, Enterobacter and Staphylococcus produce beta-lactamase enzyme. Beta-lactamase enzyme breaks the beta-lactam ring in cephalosporins open and therefore, impedes them to induce bacterial effect. As a result, the patient cannot be treated by antibiotics administration.

In order to prevent bacterial infections and preclude the bacteria to inactivate the antibiotics, it has been alternatively developed that penicillins or cephalosporins are combined with beta lactamase enzyme inhibitors. Clavulanic acid which is a beta-lactamase inhibitor is a molecule with weak antibacterial activity that was disclosed in the patent numbered DE 2517316.

Oral solid dosage forms comprising cephalosporin antibiotics and clavulanic acid are not preferred to be administered especially by the patients having difficulty in swallowing. Suspension forms are also not preferable since they have the potential to cause high and/or uncontrolled dose intake and have difficulty for using and carrying.

Alternatively developed water soluble formulations like effervescent formulations comprising cephalosporin antibiotics and clavulanic acid are used to overcome the problems seen in the solid oral dosage forms and suspension forms.

However, some problems have been confronted during the preparation of the effervescent formulations comprising the combination of cephalosporin antibiotics and clavulanic acid. During the process for their preparations, when cephalosporin antibiotic contact with water, gelling or agglomeration problems are seen due to the fact that cephalosporin antibiotics are hydrophobic molecules and thus they have low solubility in aqueous media. Furthermore, stability problem of clavulanic acid is observed when it contacts with water and some of the commonly used excipients, since clavulanic acid is sensitive to some environmental effects such as moisture and pH.

Therefore, it is difficult to formulate effervescent formulations comprising the combination of cephalosporin antibiotics and clavulanic acid during the process for the preparation thereof due to low water solubility of cephalosporins and instability of clavulanic acid in aqueous media.

As is seen, there is a requirement for development of new preparation methods for effervescent formulations comprising the combination wherein gelling or agglomeration problem resulting from low solubility of cefdinir and also stability problem of clavulanic acid are eliminated during their preparation.

### SUMMARY OF THE INVENTION

In one aspect, the invention features a process for the preparation of effervescent formulation including a cephalosporin antibiotic and clavulanic acid, or pharmaceutically acceptable derivatives thereof, characterized in that the cephalosporin antibiotic and clavulanic acid are mixed in the presence of high molecular weight PEG with the granules including, e.g., effervescent couple and at least one excipient. This process for the preparation of the effervescent formulation can include, e.g.,:
I. granulating the effervescent acid, effervescent base, and at least one other pharmaceutically acceptable excipient by a solvent,
II. drying and sieving the obtained granules (e.g., below the temperature of 100° C.),
III. adding cephalosporin antibiotic, clavulanic acid, high molecular weight PEG, and optionally at least one other pharmaceutically acceptable excipient into the granules that are obtained in step II,
IV. optionally compressing the final mixture obtained in step III into tablets or filling the final mixture into sachets.

In the above process, the granules can, e.g., be dried in such a way that moisture ratio is in the range of 0.1-2%.

In another aspect, the invention features a formulation prepared as described above, where the formulation includes a cephalosporin antibiotic and clavulanic acid, or pharmaceutically acceptable derivatives thereof, high molecular weight PEG (e.g., PEG 4000, PEG 6000, or a combination thereof), effervescent couple, and at least one other pharmaceutically acceptable excipient.

In the formulations of the invention, the cephalosporin antibiotic can be, e.g., cefazolin, cefacetrile, cephadroxyl, cephalexin, cephaloglycin, cefalonium, cephaloridine, cephalothin, cephaprin, cefatrizine, cefazedone, cefazaflur, cefradine, cefroxadine, ceftezole, cefaclor, cefamandole, cefminox, cefocinid, ceforanide, cefotiam, cefprozil, cefbuperazone, cefuroxime, cefuzonam, cephamycin (cefoxitin, cefotetan, cefmetazole), carbacephem (loracarbef), cefixime, ceftazidime, ceftriaxone, cefcapene, cefdaloxime, cefdinir, cefditoren, cefetamet, cefmenoxime, cefodizime, cefoperazone, cefotaxime, cefpimizole, cefpiramide, cefpodoxime, cefsulodin, cefteram, ceftibuten, ceftiolene, ceftizoxime, oxacephem (flomoxef, latamoxef), cefepime, cefozopran, cefpirome, cefquinome, ceftobiprole, ceftiofur, cefquinome, cefovecin, or a pharmaceutically acceptable derivative thereof.

The cephalosporin antibiotic and/or clavulanic acid used in the formulation can be, e.g., in the form of their pharmaceutically acceptable hydrates, solvates, esters, enantiomers, crystalline forms, amorphous forms, salt forms (e.g., sodium, potassium, calcium, magnesium, aluminum, ammonium, and modified ammonium salt form) or free base form, and/or a combination thereof.

In certain embodiments, the cephalosporin antibiotic can be in the range of 15-40% by weight (e.g., 1-40% by weight) and/or be present in the range of 100-1500 mg (e.g., 250-800 mg, 300-800 mg, or 100-1400 mg) by weight). The clavulanic acid can be, e.g., in the range of 5-25% by weight and/or in an amount in the range of 50-450 mg (e.g., 50-400 mg). The high molecular weight PEG can be, e.g., in an amount in the range of 0.2-5% by weight. The effervescent couple can be, e.g., present in a range of 20-70% by weight. The effervescent acid can be, e.g., in the range of 5-50% by weight, the effervescent base can be in the range of 5-45% by weight. The remaining pharmaceutically acceptable excipients can be, e.g., present in the range of 1-20% by weight. For example, a binder can be in the range of 0.5-5% by weight, a sweetener can be in the range of 1-4% by weight, a flavoring agent can be in the range of 0.1-5% by weight, and/or a coloring agent can be in the range of 0.5-4% by weight.

In certain formulations of the invention, the effervescent couple can include an effervescent acid (e.g., acetic acid, citric acid, lactic acid, malic acid, phosphoric acid, propionic acid, and tartaric acid, or combinations thereof) and an effervescent base (e.g., potassium carbonate, potassium bicarbonate, potassium citrate, potassium hydroxide, sodium carbonate, sodium hydrogen carbonate, and sodium hydrogen citrate, or combinations thereof).

Other pharmaceutically acceptable excipients that can be, e.g., used in the formulations of the invention include binders, glidants, diluents, disintegrants, flavoring agents, sweeteners, coloring agents, anti-foam agent, and stabilizing agents.

In some embodiments, the binders used in the formulation can be a starch, such as, e.g., potato starch, corn starch, wheat starch; a sugar, such as, e.g., sucrose, glucose, dextrose, lactose, maltodextrin; natural and synthetic gums; gelatin; a cellulose derivative, such as, e.g., microcrystalline cellulose, HPC, HEC, HPMC, carboxymethyl cellulose, methyl cellulose, ethyl cellulose; polyvinylpyrrolidone (povidone), polyethylene glycol (PEG); waxes; calcium carbonate; calcium phosphate; an alcohol such as, e.g., sorbitol, xylitol, mannitol, water, or a combination thereof.

In formulations including a flavoring agent, such flavoring agents can be, e.g., natural aroma oils, menthol, menthane, anethole, methyl salicylate, eucalyptol, cinnamon, 1-methyl acetate, sage, eugenol, oxanone, alpha irisone, marjoram, lemon, orange, blackberry, propenyl guaetol acetyl, cinnamon, vanilla, thymol, linalol, cinnamaldehyde glycerol acetal, or a combination thereof.

In formulations including a sweetener, the sweetener can be, e.g., sucralose, sucrose, fructose, glucose, galactose, xylose, dextrose, laevulose, lactose, maltose, maltodextrin, mannitol, maltitol, maltol, sorbitol, xylitol, erythritol, lactitol, isomalt, corn syrup, saccharine, saccharine salts, acesulfame potassium, aspartame, D, cyclamates, or a combination thereof.

In formulation including a coloring agent, the coloring agent can be In formulations including a carotenoids, chlorophyl, or a combination thereof.

### DESCRIPTION OF THE INVENTION

The inventors have surprisingly found that the problems in the prior art can be solved by the process according to present invention used for the preparation of effervescent formulations comprising cephalosporins and clavulanic acid or any pharmaceutically acceptable derivative thereof.

The present invention relates to the process for the preparation of the effervescent formulations comprising a cephalosporin antibiotic and clavulanic acid or any pharmaceutically acceptable derivative thereof and their preparation methods. The inventors have surprisingly found that when cephalosporin antibiotic and clavulanic acid are mixed with the granules comprising effervescent couple and at least one excipient in the presence of high-molecular weight PEG, gelling or agglomeration problem of cefdinir and also stability problem of clavulanic acid are eliminated.

The first aspect of the present invention is the process for the preparation of the effervescent formulations comprising a cephalosporin antibiotic and clavulanic acid or any pharmaceutically acceptable derivative thereof wherein the cephalosporin antibiotic and clavulanic acid are mixed in the presence of high-molecular weight PEG with the granules comprising effervescent couple and at least one excipient.

Cephalosporin antibiotic used in the effervescent formulation prepared by the process according to the present invention can be selected from a group comprising cefazolin, cefacetrile, cephadroxyl, cephalexin, cephaloglycin, cefalonium, cephaloridine, cephalothin, cephaprin, cefatrizine, cefazedone, cefazaflur, cefradine, cefroxadine, ceftezole, cefaclor, cefamandole, cefminox, cefocinid, ceforanide, cefotiam, cefprozil, cefbuperazone, cefuroxime, cefuzonam, cephamycin (cefoxitin, cefotetan, cefmetazole), carbacephem (loracarbef), cefixime, ceftazidime, ceftriaxone, cefcapene, cefdaloxime, cefdinir, cefditoren, cefetamet, cefmenoxime, cefodizime, cefoperazone, cefotaxime, cefpimizole, cefpiramide, cefpodoxime, cefsulodin, cefteram, ceftibuten, ceftiolene, ceftizoxime, oxacephem (flomoxef, latamoxef), cefepime, cefozopran, cefpirome, cefquinome, ceftobiprole, ceftiofur, cefquinome, cefovecin or any pharmaceutically acceptable derivative thereof.

The cephalosporin antibiotic that is used in the effervescent formulation prepared by the process according to the present invention is preferably selected from the group comprising cefaclor, cefprozil, cefuroxime, cefdinir, cefditoren, cefetamet, ceftibuten or a pharmaceutically acceptable derivative thereof.

The cephalosporin antibiotic that is used in the effervescent formulation prepared by the process according to the present invention is more preferably selected from the group comprising cefaclor, cefprozil, cefuroxime and cefetamet or any pharmaceutically acceptable derivative thereof.

The term “pharmaceutically acceptable derivative” in “a cephalosporin antibiotic and clavulanic acid or any pharmaceutically acceptable derivative” means that the cephalosporin antibiotic and/or the clavulanic acid can be in the form of its pharmaceutically acceptable hydrates, solvates, esters, enantiomers, crystal forms, amorphous forms, salt forms or free base form and/or a combination thereof.

The formulation prepared by the process according to the present invention comprises a cephalosporin antibiotic in the range of 15-40% by weight.

The formulation prepared by the process according to the present invention comprises a cephalosporin antibiotic in the range of 100-1500 mg, preferably in the range of 250-800 mg or a pharmaceutically acceptable derivative thereof in an equal amount.

Clavulanic acid that is used in the effervescent formulation prepared by the process according to the present invention can be in pharmaceutically acceptable salt form, preferably in sodium, potassium, calcium, magnesium, aluminum, ammonium and modified ammonium salt form.

Clavulanic acid that is used in the pharmaceutical formulation prepared by the process according to the present invention is preferably in potassium or sodium salt form.

Clavulanic acid that is used in the pharmaceutical formulation prepared by the process according to the present invention is preferably potassium clavulanate.

The formulation prepared by the process according to the present invention comprises 5-25% clavulanic acid by weight.

The formulation prepared by the process according to the present invention comprises clavulanic acid in the range of 50-450 mg, preferably in the range of 50-250 mg, preferably in the range of 65-125 mg or a pharmaceutically acceptable derivative thereof in an equal amount.

The effervescent formulation prepared by the process in accordance with the process of the present invention can be stored in tablet and/or sachet form.

High molecular weight PEG that is used in the effervescent formulation prepared by the process according to the present invention can be PEG 6000 or PEG 4000 or a combination thereof. Preferably, PEG 6000 is used as high molecular weight PEG.

High molecular weight PEG that is used in the effervescent formulation prepared by the process according to the present invention is in the range of 0.2-5% by weight.

The process for the preparation of the effervescent formulations comprising a cephalosporin antibiotic and clavulanic acid, which is the subject of the invention, includes the following steps of;
I. granulating effervescent acid, effervescent base, and at least one other excipient by a solvent
II. drying and sieving the obtained granules III. adding cephalosporin antibiotic, clavulanic acid, high molecular weight PEG, and optionally at least one other pharmaceutically acceptable excipient into the granules that are obtained in step II.
IV. optionally compressing the final mixture obtained in step III into tablets or filling it into sachets.

In the first step of the process in accordance with the invention, the effervescent couple, sweetener and binder are granulated with the water. Therefore, only the effervescent couple and the excipients contact with water; cephalosporin antibiotic and clavulanic acid do not contact with water.

In the second step of the process according to the present invention, the granules obtained in the first step are dried below the temperature of 100° C., preferably in the range of 30-90° C., more preferably 45-70 ° C. in such a way that the moisture ratio of them will be in the range of 0.1-2%, preferably 0.2-1%.

In the third step of the process according to the present invention, the cephalosporin antibiotic and clavulanic acid are added to the obtained granules in the presence of high molecular weight PEG. In other words, the active agents, which are cephalosporin antibiotic and clavulanic acid or pharmaceutically acceptable derivatives thereof, are added to the process extragranularly and do not contact with water. Accordingly, stable and homogeneously soluble effervescent formulations comprising cephalosporin antibiotic and clavulanic acid are obtained.

The effervescent formulation prepared by the process according to the present invention can comprise pharmaceutically acceptable excipients in addition to the active agent cephalosporin antibiotic, clavulanic acid and a high molecular weight PEG.

The effervescent formulation prepared by the process according to the present invention can optionally comprise one or more of the excipients including effervescent acid, effervescent base, binders, glidants, diluents, disintegrants, flavoring agents, sweeteners, coloring agents, anti-foam agent, stabilizing agents.

The pharmaceutically acceptable effervescent acid used in the formulation prepared by the process according to the present invention can be selected from a group comprising acetic acid, citric acid, lactic acid, malic acid, phosphoric acid, propionic acid, tartaric acid or combinations thereof.

The pharmaceutically acceptable effervescent base used in the formulation prepared by the process according to the present invention can be selected from a group comprising potassium carbonate, potassium bicarbonate, potassium citrate, potassium hydroxide, sodium carbonate, sodium hydrogen carbonate, sodium hydrogen citrate or combinations thereof. The binder that is used in the formulation prepared by the process according to the present invention can be selected from a group comprising starches such as potato starch, corn starch, wheat starch; sugars such as sucrose, glucose, dextrose, lactose, maltodextrin; natural and synthetic gums; gelatin; cellulose derivatives such as microcrystalline cellulose, HPC, HEC, HPMC, carboxymethyl cellulose, methyl cellulose, ethyl cellulose; polyvinylpyrrolidone (povidone), polyethylene glycol (PEG); waxes; calcium carbonate; calcium phosphate; alcohols such as sorbitol, xylitol, mannitol, and water or a combination thereof.

The flavoring agent that is used in the formulation prepared by the process according to the present invention can be selected from a group comprising natural aroma oils, menthol, menthane, anethole, methyl salicylate, eucalyptol, cinnamon, 1-methyl acetate, sage, eugenol, oxanone, alpha irisone, marjoram, lemon, orange, blackberry, propenyl guaetol acetyl, cinnamon, vanilla, thymol, linalol, cinnamaldehyde glycerol acetal or a combination thereof.

The sweetener that is used in the formulation prepared by the process according to the present invention can be selected from a group comprising sucralose, sucrose, fructose, glucose, galactose, xylose, dextrose, laevulose, lactose, maltose, maltodextrin, mannitol, maltitol, maltol, sorbitol, xylitol, erythritol, lactitol, isomalt, corn syrup, saccharine, saccharine salts, acesulfame potassium, aspartame, D and cyclamates or a combination thereof.

The coloring agent that is used in the formulation prepared by the process according to the present invention can be selected from a group comprising carotenoids and chlorophyl or a combination thereof.

The glidant that can be used in the formulation prepared by the process according to the present invention can be selected from a group comprising sodium benzoate, sodium chloride, sodium acetate, sodium fumarate, carbowax 4000, L-leucine, PEG or a combination thereof.

The diluent that can be used in the formulation prepared by the process according to the present invention can be selected from a group comprising lactose, maltose, dextrin, maltodextrin, mannitol, sorbitol, starch or a combination thereof.

The disintegrant that can be used in the formulation prepared by the process according to the present invention can be selected from a group comprising starches such as potato starch, corn starch, wheat starch, pregelatinized starch, sodium starch glycolate; cellulose derivatives such as croscarmellose sodium or microcrystalline cellulose; polyvinylpyrrolidone; crospovidone; alginic acid and its salts; clays such as xanthan gum or veegum; ion exchange resins or a combination thereof.

In another aspect, the process for the preparation of the effervescent formulations comprising a cephalosporin antibiotic and clavulanic acid, which is the subject of the invention, includes the following steps of;
I. granulating effervescent acid, effervescent base, sweetener and binder by a solvent
II. drying and sieving the obtained granules
III. adding cephalosporin antibiotic, clavulanic acid, high molecular weight PEG, flavoring agent and coloring agent into the granules that are obtained in step II.
IV. optionally compressing the final mixture obtained in step III into tablets or fillilng them into sachets.

The effervescent formulation prepared by the process according to the process of the present invention comprises; a cephalosporin antibiotic in the range of 15-40% by weight, clavulanic acid or pharmaceutically acceptable derivatives thereof in the range of 5-25% by weight, high molecular weight PEG in the range of 0.2-5% by weight, an effervescent acid in the range of 5-50% by weight, an effervescent base in the range of 5-45% by weight, binder in the range of 0.5-5% by weight, sweetener in the range of 1-4%, flavoring agent in the range of 0.1-5% by weight and coloring agent in the range of %0.5-4% by weight.

In another aspect, the present invention relates to use of the effervescent formulation in upper respiratory tract infections such as pharyngitis, tonsillitis, otitis media, sinusitis; lower respiratory tract infections such as chronic bronchitis and pneumonia; skin and soft tissue infections, urinary system infections and gonorrhea.

The effervescent formulations according to the process of the present invention can be prepared as described below, but not limited to these examples.

### Example 1

### The Formulation and the Process for the Preparation of the Effervescent Tablet

| Component | Amount (% weight) |
|---|---|
| Cefaclor | 20.5 |
| Potassium clavulanate | 12 |
| Effervescent acid | 32 |
| Effervescent base | 27 |
| PEG 6000 | 1.5 |
| Binder | 2.5 |
| Sweetener | 2 |
| Flavoring agent | 1.5 |
| Coloring agent | 1 |

The process for the preparation of the effervescent formulation comprising cefaclor and potassium clavulanate comprises the steps of;
granulating effervescent acid, effervescent base, sweetener and binder by water
drying and sieving the obtained granules;
adding cefaclor, potassium clavulanate, PEG 6000, flavoring agent and coloring agent into the obtained granules and
finally compressing the final mixture into tablets.

### Example 2

### The Formulation and the Process for the Preparation of the Effervescent Powder/Granules

| Component | Amount (% weight) |
|---|---|
| Cefprozil | 22 |
| Potassium clavulanate | 10 |
| Effervescent acid | 34 |
| Effervescent base | 25.5 |
| PEG 6000 | 1.5 |
| Binder | 2 |
| Sweetener | 2.5 |
| Flavoring agent | 1 |
| Coloring agent | 1.5 |

The process for the preparation of the effervescent formulation comprising cefprozil and potassium clavulanate comprises the steps of;
granulating effervescent acid, effervescent base, sweetener and binder by water;
drying and sieving the obtained granules;
adding cefprozil, potassium clavulanate, PEG 6000, flavoring agent and coloring agent into the obtained granules and
finally filling the final mixture into sachets.

### Example 3

### The Formulation and the Process for the Preparation of the Effervescent Powder/Granules

| Component | Amount (% weight) |
|---|---|
| Cefuroxime axetil | 20 |
| Potassium clavulanate | 9 |
| Effervescent acid | 30.5 |
| Effervescent base | 32 |
| PEG 6000 | 1.2 |
| Binder | 2.8 |
| Sweetener | 2 |
| Flavoring agent | 1.5 |
| Coloring agent | 1 |

The process for the preparation of the effervescent formulation comprising cefuroxime axetil and potassium clavulanate comprises the steps of;
granulating effervescent acid, effervescent base, sweetener and binder by water;
drying and sieving the obtained granules;
adding cefuroxime axetil, potassium clavulanate, PEG 6000, flavoring agent and coloring agent into the obtained granules and
finally filling the final mixture into sachets.

### Example 4

### The Formulation and the Process for the Preparation of the Effervescent Tablet

| Component | Amount (% weight) |
|---|---|
| Cefdinir | 24.5 |
| Potassium clavulanate | 11 |
| Effervescent acid | 31.5 |
| Effervescent base | 24 |
| PEG 6000 | 1.5 |
| Binder | 2.7 |
| Sweetener | 1.3 |
| Flavoring agent | 2 |
| Coloring agent | 1.5 |

The process for the preparation of the effervescent formulation comprising cefdinir and potassium clavulanate comprises the steps of;
granulating effervescent acid, effervescent base, sweetener and binder by water
drying and sieving the obtained granules;
adding cefdinir, potassium clavulanate, PEG 6000, flavoring agent and coloring agent into the obtained granules and
finally compressing the final mixture into tablets.

### Example 5

### The Formulation and the Process for the Preparation of the Effervescent Tablet

| Component | Amount (% weight) |
|---|---|
| Cefditoren | 30.5 |
| Potassium clavulanate | 14 |
| Effervescent acid | 27 |
| Effervescent base | 21.6 |
| PEG 6000 | 1.4 |
| Binder | 0.8 |
| Sweetener | 2.2 |
| Flavoring agent | 1.5 |
| Coloring agent | 1 |

The process for the preparation of the effervescent formulation comprising cefditoren and potassium clavulanate comprises the steps of;
granulating effervescent acid, effervescent base, sweetener and binder by water
drying and sieving the obtained granules;
adding cefditoren, potassium clavulanate, PEG 6000, flavoring agent and coloring agent into the obtained granules and
finally compressing the final mixture into tablets.

### Example 6

### The Formulation and the Process for the Preparation of the Effervescent Tablet

| Component | Amount (% weight) |
|---|---|
| Ceftibuten | 28 |
| Potassium clavulanate | 16 |
| Effervescent acid | 27.5 |
| Effervescent base | 21.5 |
| PEG 6000 | 1 |
| Binder | 2 |
| Sweetener | 1.9 |
| Flavoring agent | 1.1 |
| Coloring agent | 1 |

The process for the preparation of the effervescent formulation comprising ceftibuten and potassium clavulanate comprises the steps of;
granulating effervescent acid, effervescent base, sweetener and binder by water
drying and sieving the obtained granules;
adding ceftibuten, potassium clavulanate, PEG 6000, flavoring agent and coloring agent into the obtained granules and
finally compressing the final mixture into tablets.

### Example 7

### The Formulation and the Process for the Preparation of the Effervescent Powder/Granules

| Component | Amount (% weight) |
|---|---|
| Cefetamet | 24 |
| Potassium clavulanate | 15 |
| Effervescent acid | 32 |
| Effervescent base | 22 |
| PEG 6000 | 0.7 |
| Binder | 1.5 |
| Sweetener | 2 |
| Flavoring agent | 1.3 |
| Coloring agent | 1.5 |

The process for the preparation of the effervescent formulation comprising cefetamet and potassium clavulanate comprises the steps of;
granulating effervescent acid, effervescent base, sweetener and binder by water;
drying and sieving the obtained granules;
adding cefetamet, potassium clavulanate, PEG 6000, flavoring agent and coloring agent into the obtained granules and
   finally filling the final mixture into sachets.

The present invention also relates to the effervescent formulations comprising a cephalosporin antibiotic and clavulanic acid or any pharmaceutically acceptable derivative thereof and their preparation methods. Surprisingly, it is seen that effervescent forms formulated with the formulation comprising 10-40% of a cephalosporin antibiotic, 5-25% clavulanic acid, 20-70% effervescent couple, 0.2-5% high molecular weight PEG and %1-20 other pharmaceutically acceptable excipients with respect to the total weight of unit dose, homogeneously and quickly dissolve in water and are stable.

The first aspect of the present invention is the effervescent formulations comprising a cephalosporin antibiotic and clavulanic acid or any pharmaceutically acceptable derivative thereof wherein the formulation comprises 10-40% of a cephalosporin antibiotic, 5-25% clavulanic acid, 20-70% effervescent couple, 0.2-5% high molecular weight PEG and %1-20 other pharmaceutically acceptable excipients with respect to the total weight of unit dose.

This way it was observed that the clavulanic acid or any pharmaceutically acceptable derivative thereof remains stable due to the fact that the composition is kept in solid form. Furthermore the composition is dissolved in water completely and homogeneously prior to administration owing to the use of active agents, the effervescent couple, high molecular weight PEG and other excipients in the amounts given above.

The term “total weight of unit dose” comprises the weight of a cephalosporin antibiotic, clavulanic acid, high molecular weight PEG, effervescent couple and the other pharmaceutically acceptable excipients.

The effervescent formulation of the present invention can be in granule, powder or tablet form. Preferably, it is in tablet form.

When the effervescent formulations comprising cephalosporin antibiotic and clavulanic acid are formulated in effervescent tablet form, it is observed that the final tablet forms can dissolve in water in less than 5 minutes which is a short time for a drug to dissolve before administration.

Accordingly, another aspect of the present invention is the effervescent tablet forms comprising a cephalosporin antibiotic and clavulanic acid which dissolve in water less than 5 minutes.

The cephalosporin antibiotic used in the effervescent formulation of the present invention can be selected from a group comprising cefazolin, cefacetrile, cephadroxyl, cephalexin, cephaloglycin, cefalonium, cephaloridine, cephalothin, cephaprin, cefatrizine, cefazedone, cefazaflur, cefradine, cefroxadine, ceftezole, cefaclor, cefamandole, cefminox, cefocinid, ceforanide, cefotiam, cefprozil, cefbuperazone, cefuroxime, cefuzonam, cephamycin (cefoxitin, cefotetan, cefmetazole), carbacephem (loracarbef), cefixime, ceftazidime, ceftriaxone, cefcapene, cefdaloxime, cefdinir, cefditoren, cefetamet, cefmenoxime, cefodizime, cefoperazone, cefotaxime, cefpimizole, cefpiramide, cefpodoxime, cefsulodin, cefteram, ceftibuten, ceftiolene, ceftizoxime, oxacephem (flomoxef, latamoxef), cefepime, cefozopran, cefpirome, cefquinome, ceftobiprole, ceftiofur, cefquinome, cefovecin or any pharmaceutically acceptable derivative thereof.

The cephalosporin antibiotic that is used in the effervescent formulation of the present invention is preferably selected from the group comprising cefaclor, cefprozil, cefuroxime, cefdinir, cefditoren, cefetamet, ceftibuten or a pharmaceutically acceptable derivative thereof.

The cephalosporin antibiotic that is used in the effervescent formulation of the present invention is more preferably selected from the group comprising cefaclor, cefprozil, cefuroxime and cefetamet or any pharmaceutically acceptable derivative thereof.

The cephalosporin antibiotic that is used in the effervescent formulation of the present invention can be in the form of its pharmaceutically acceptable hydrates, solvates, esters, enantiomers, polymorphs, crystal forms, amorphous forms, salt forms or free base form and/or a combination thereof.

The formulation of the present invention comprises a cephalosporin antibiotic in the range of 100-1400 mg, preferably in the range of 300-800 mg or a pharmaceutically acceptable derivative thereof in an equal amount.

The formulation of the present invention comprises a cephalosporin antibiotic preferably in the range of 15-35%, more preferably 20-30% by weight with respect to the total weight of unit dose.

Clavulanic acid that the effervescent formulation of the present invention comprises can be in the form of its pharmaceutically acceptable hydrates, solvates, esters, enantiomers, polymorphs, crystal forms, amorphous forms, salt forms or free base form and/or a combination thereof.

Clavulanic acid that the pharmaceutical formulation of the present invention comprises can be in salt form, preferably in sodium, potassium, calcium, magnesium, aluminum, ammonium and modified ammonium salt form.

Clavulanic acid comprised in the pharmaceutical formulation of the present invention is preferably potassium clavulanate.

The formulation of the present invention comprises clavulanic acid in the range of 50-400 mg, preferably in the range of 50-300 mg, preferably in the range of 70-125 mg or a pharmaceutically acceptable derivative thereof in an amount equivalent to that.

The formulation of the present invention comprises clavulanic acid or a pharmaceutically acceptable derivative thereof in the range of 8-22%, more preferably 10-20% by weight with respect to the total weight of unit dose.

High molecular weight PEG that is used in the effervescent formulation of the present invention can be PEG 6000 or PEG 4000 or a combination thereof.

The formulation of the present invention comprises high molecular weight PEG preferably in the range of 0.3-3%, more preferably 0.5-2% by weight with respect to the total weight of unit dose.

The effervescent couple comprises an effervescent acid and an effervescent base.

The pharmaceutically acceptable effervescent acid of the present invention can be selected from a group comprising acetic acid, citric acid, lactic acid, malic acid, phosphoric acid, propionic acid, tartaric acid or combinations thereof.

The pharmaceutically acceptable effervescent base of the present invention can be selected from a group comprising potassium carbonate, potassium bicarbonate, potassium citrate, potassium hydroxide, sodium carbonate, sodium hydrogen carbonate, sodium hydrogen citrate or combinations thereof.

The effervescent formulation of the present invention comprises other pharmaceutically acceptable excipients in addition to the active agent cephalosporin antibiotic, clavulanic acid, effervescent couple and high molecular weight PEG.

The effervescent formulation of the present invention comprise one or more of the other pharmaceutically excipients selected from the group comprising binders, glidants, diluents, disintegrants, flavoring agents, sweeteners, coloring agents, anti-foam agent, stabilizing agents. The binder that is used in the formulation according to the present invention can be selected from a group comprising starches such as potato starch, corn starch, wheat starch; sugars such as sucrose, glucose, dextrose, lactose, maltodextrin; natural and synthetic gums; gelatin; cellulose derivatives such as microcrystalline cellulose, HPC, HEC, HPMC, carboxymethyl cellulose, methyl cellulose, ethyl cellulose; polyvinylpyrrolidone (povidone), polyethylene glycol (PEG); waxes; calcium carbonate; calcium phosphate; alcohols such as sorbitol, xylitol, mannitol, and water or a combination thereof.

The flavoring agent that is used in the formulation according to the present invention can be selected from a group comprising natural aroma oils, menthol, menthane, anethole, methyl salicylate, eucalyptol, cinnamon, 1-methyl acetate, sage, eugenol, oxanone, alpha irisone, marjoram, lemon, orange, blackberry, propenyl guaetol acetyl, cinnamon, vanilla, thymol, linalol, cinnamaldehyde glycerol acetal or a combination thereof.

The sweetener that is used in the formulation according to the present invention can be selected from a group comprising sucralose, sucrose, fructose, glucose, galactose, xylose, dextrose, laevulose, lactose, maltose, maltodextrin, mannitol, maltitol, maltol, sorbitol, xylitol, erythritol, lactitol, isomalt, corn syrup, saccharine, saccharine salts, acesulfame potassium, aspartame, D and cyclamates or a combination thereof.

The coloring agent that is used in the formulation according to the present invention can be selected from a group comprising carotenoids and chlorophyl or a combination thereof.

The glidant that can be used in the formulation according to the present invention can be selected from a group comprising sodium benzoate, sodium chloride, sodium acetate, sodium fumarate, carbowax 4000, L-leucine, PEG or a combination thereof.

The diluents that can be used in the formulation according to the present invention can be selected from a group comprising lactose, maltose, dextrin, maltodextrin, mannitol, sorbitol, starch or a combination thereof.

The disintegrant that can be used in the formulation according to the present invention can be selected from a group comprising starches such as potato starch, corn starch, wheat starch, pregelatinized starch, sodium starch glycolate; cellulose derivatives such as croscarmellose sodium or microcrystalline cellulose; polyvinylpyrrolidone; crospovidone; alginic acid and its salts; clays such as xanthan gum or veegum; ion exchange resins or a combination thereof.

The antifoam agent that can be used in the formulation according to the present invention can be selected from a group comprising simethicone emulsion and dimethyl siloxane, silicon oil or a combination thereof.

The stabilizing agent and/or agents that can be used in the formulation according to the present invention can be selected from a group comprising antioxidants, chelating agents, alkalinizing agents and photoprotective agents.

In another aspect, the present invention relates to the process for the preparation of the effervescent formulations comprising a cephalosporin antibiotic and clavulanic acid includes the following steps of granulating effervescent couple and at least one excipient; adding cephalosporin antibiotic, clavulanic acid and the other excipients to the obtained granules and optionally compressing the final mixture into tablets.

In another aspect, the present invention relates to use of the effervescent formulation in upper respiratory tract infections such as pharyngitis, tonsillitis, otitis media, sinusitis; lower respiratory tract infections such as chronic bronchitis and pneumonia; skin and soft tissue infections, urinary system infections and gonorrhea.

The effervescent formulations pertaining to the present invention can be prepared as described below, but not limited to these examples.

### Example 8

### The Formulation of the Effervescent Tablet and its Preparation

| Component | Amount (% weight) |
|---|---|
| Cefaclor | 27 |
| Potassium clavulanate | 15 |
| Effervescent couple | 48.5 |
| PEG 6000 | 1.5 |
| Other excipient | 8 |

The effervescent formulation comprising cefaclor and potassium clavulanate is prepared by granulating effervescent couple and at least one excipient; adding cefaclor, potassium clavulanate and the other excipients to the obtained granules and finally compressing the final mixture into tablets.

### Example 9

### The Formulation of the Effervescent Granule/Powder and its preparation

| Component | Amount (% weight) |
|---|---|
| Cefprozil | 23.5 |
| Potassium clavulanate | 16 |
| Effervescent couple | 51 |
| PEG 6000 | 1 |
| Other excipient | 8.5 |

The effervescent formulation comprising cefprozil and potassium clavulanate is prepared by granulating effervescent couple and at least one excipient; adding cefprozil, potassium clavulanate and the other excipients to the obtained granules and finally filling the final mixture into sachets.

### Example 10

### The Formulation of the Effervescent Tablet and its Preparation

| Component | Amount (% weight) |
|---|---|
| Cefdinir | 22 |
| Potassium clavulanate | 16 |
| Effervescent couple | 57 |
| PEG 6000 | 1.5 |
| Other excipient | 3.5 |

The effervescent formulation comprising cefdinir and potassium clavulanate is prepared by granulating effervescent couple and at least one excipient; adding cefdinir, potassium clavulanate and the other excipients to the obtained granules and finally compressing the final mixture into tablets.

### Example 11

### The Formulation of the Effervescent Granule/Powder and its Preparation

| Component | Amount (% weight) |
|---|---|
| Cefuroxime axetil | 26 |
| Potassium clavulanate | 12 |
| Effervescent couple | 53 |
| PEG 6000 | 1 |
| Other excipient | 8 |

The effervescent formulation comprising cefuroxime axetil and potassium clavulanate is prepared by granulating effervescent couple and at least one excipient; adding cefuroxime axetil, potassium clavulanate and the other excipients to the obtained granules and finally filling the final mixture into sachets.

### Example 12

### The Formulation of the Effervescent Tablet and its Preparation

| Component | Amount (% weight) |
|---|---|
| Ceftibuten | 27.5 |
| Potassium clavulanate | 12.5 |
| Effervescent couple | 55 |
| PEG 6000 | 1.3 |
| Other excipient | 3.7 |

The effervescent formulation comprising ceftibuten and potassium clavulanate is prepared by granulating effervescent couple and at least one excipient; adding ceftibuten, potassium clavulanate and the other excipients to the obtained granules and finally compressing the final mixture into tablets.

### Example 13

### The Formulation of the Effervescent Tablet and its Preparation

| Component | Amount (% weight) |
|---|---|
| Cefditoren | 28 |
| Potassium clavulanate | 11.8 |
| Effervescent couple | 54 |
| PEG 6000 | 1.2 |
| Other excipient | 5 |

The effervescent formulation comprising cefditoren and potassium clavulanate is prepared by granulating effervescent couple and at least one excipient; adding cefditoren, potassium clavulanate and the other excipients to the obtained granules and finally compressing the final mixture into tablets.

### Example 14

### The Formulation of the Effervescent Granule/Powder and its Preparation

| Component | Amount (% weight) |
|---|---|
| Cefetamet | 25 |
| Potassium clavulanate | 13 |
| Effervescent couple | 53 |
| PEG 6000 | 1.75 |
| Other excipient | 7.25 |

The effervescent formulation comprising cefetamet and potassium clavulanate is prepared by granulating effervescent couple and at least one excipient; adding cefetamet, potassium clavulanate and the other excipients to the obtained granules and finally filling the final mixture into sachets.
free

What is claimed is:

1. A process for the preparation of an effervescent formulation comprising a cephalosporin antibiotic and clavulanic acid or pharmaceutically acceptable derivatives thereof characterized in that the cephalosporin antibiotic and clavulanic acid are mixed in the presence of high molecular weight PEG with granules comprising effervescent couple and at least one excipient.

2. The process for the preparation of the effervescent formulation comprising a cephalosporin antibiotic and clavulanic acid or pharmaceutically acceptable derivatives thereof according to claim 1, wherein said process comprises the following steps of;
I. granulating effervescent acid, effervescent base, and at least one other pharmaceutically acceptable excipient by a solvent,
II. drying and sieving the obtained granules,
III. adding cephalosporin antibiotic, clavulanic acid, high molecular weight PEG, and optionally at least one other pharmaceutically acceptable excipient into the granules that are obtained in step II, and
IV. optionally compressing the final mixture obtained in step III into tablets or filling the final mixture into sachets.

3. The process for the preparation of the effervescent formulation according to claim 2 wherein the granules are dried below a temperature of 100° C.

4. The process for the preparation of the effervescent formulation according to claim 2 wherein the granules are dried in such a way that moisture ratio is in the range of 0.1-2%.

5-7. (canceled)

8. An effervescent formulation prepared according to claim 1, wherein the cephalosporin antibiotic used in said formulation is selected from the group consisting of cefaclor, cefprozil, cefuroxime, and cefetamet, or any pharmaceutically acceptable derivative thereof.

9-11. (canceled)

12. An effervescent formulation prepared according to claim 1, wherein the cephalosporin antibiotic is used in the range of 250-800 mg by weight.

13-15. (canceled)

16. An effervescent formulation prepared according to claim 1, wherein clavulanic acid is potassium clavulanate.

17. (canceled)

18. An effervescent formulation prepared according to claim 1, wherein clavulanic acid is in an amount in the range of 50-450 mg.

19. An effervescent formulation prepared according to claim 1, wherein high molecular weight PEG is selected from PEG 4000, PEG 6000, or a combination thereof.

20. The effervescent formulation according to claim 19, wherein high molecular weight PEG is in an amount in the range of 0.2-5% by weight.

21. (canceled)

22. An effervescent formulation prepared according to claim 1, wherein the effervescent couple comprises an effervescent acid and the effervescent acid is selected from the group consisting of acetic acid, citric acid, lactic acid, malic acid, phosphoric acid, propionic acid, and tartaric acid, and combinations thereof.

23. An effervescent formulation prepared according to claim 1, wherein the effervescent couple comprises an effervescent base and the effervescent base is selected from the group consisting of potassium carbonate, potassium bicarbonate, potassium citrate, potassium hydroxide, sodium carbonate, sodium hydrogen carbonate, and sodium hydrogen citrate, and combinations thereof.

24. An effervescent formulation prepared according to claim 1, wherein other pharmaceutically acceptable excipients are selected from the group consisting of binders, glidants, diluents, disintegrants, flavoring agents, sweeteners, coloring agents, anti-foam agent, and stabilizing agents.

25. The process for the preparation of the effervescent formulation comprising a cephalosporin antibiotic and clavulanic acid or pharmaceutically acceptable derivatives thereof according to claim 1, wherein said process comprises the following steps of;
V. granulating effervescent acid, effervescent base, sweetener and binder by a solvent
VI. drying and sieving the obtained granules
VII. adding cephalosporin antibiotic, clavulanic acid, high molecular weight PEG, flavoring agent, and coloring agent into the granules that are obtained in step II, and
VIII. compressing optionally the final mixture into tablets.

26. An effervescent formulation prepared according to claim 1, wherein said formulation comprises a cephalosporin antibiotic in the range of 15-40% by weight, clavulanic acid or pharmaceutically acceptable derivatives thereof in the range of 5-25% by weight, high molecular weight PEG in the range of 0.2-5% by weight, an effervescent acid in the range of 5-50% by weight, an effervescent base in the range of 5-45% by weight, binder in the range of 0.5-5% by weight, sweetener in the range of 1-4%, flavoring agent in the range of 0.1-5% by weight, and coloring agent in the range of 0.5-4% by weight.

27. An effervescent formulation comprising the combination of a cephalosporin antibiotic and clavulanic acid or pharmaceutically acceptable derivatives thereof characterized in that said formulation comprises;
10-40% of a cephalosporin antibiotic,
5-25% clavulanic acid,
20-70% effervescent couple,
0.2-5% high molecular weight PEG and
1-20% other pharmaceutically acceptable excipients with respect to the total weight of unit dose.

28-39. (canceled)

40. The effervescent formulation according to claim 27, wherein said formulation is formulated in granule, powder, or tablet form.

41. The effervescent formulation according to claim 40, wherein said formulation is formulated in tablet form.

42-44. (canceled)

45. The effervescent formulation according to claim 27, wherein said formulation comprises other pharmaceutically acceptable excipients in addition to the active agent cephalosporin antibiotic, clavulanic acid, effervescent couple, and high molecular weight PEG.

46-51. (canceled)
